(19) United States
(12) Patent Application Publication
(10) Pub. No.: US 2016/0150968 A1
(43) Pub. Date: Jun. 2, 2016

(54) OBJECT INFORMATION ACQUIRING APPARATUS

(71) Applicant: CANON KABUSHIKI KAISHA, Tokyo (JP)

(72) Inventors: Toru Imai, St. Louis, MO (US); Toshinobu Tokita, Yokohama-shi (JP)

(21) Appl. No.: 14/943,164

(22) Filed: Nov. 17, 2015

(30) Foreign Application Priority Data
Nov. 27, 2014 (JP) 2014-240492

(51) Int. Cl. A61B 5/00; A61B 5/107

(52) U.S. Cl. CPC A61B 5/0095; A61B 5/1075; A61B 5/742; A61B 5/489; A61B 2576/00; A61B 2562/0247; A61B 2562/0261

(57) ABSTRACT

An object information acquiring apparatus includes: a light source that generates a first light beam; a receiving unit that includes an irradiating unit emitting the first light beam so as to be directed to a predetermined optical focusing region and an acoustic wave detecting unit detecting a first acoustic wave generated when the first light beam is emitted to an object; a scanning unit that performs relative movement between the object and the receiving unit so that the receiving unit follows a concave or convex shape of a surface of the object while the first light beam is emitted and the first acoustic wave is detected; and an acquiring unit that acquires characteristics information on the optical focusing region of the object based on a detection result obtained by the acoustic wave detecting unit.

### BACKGROUND OF THE INVENTION

1. Field of the Invention

The present invention relates to an object information acquiring apparatus.

2. Description of the Related Art

In recent years, an optical imaging apparatus that allows light emitted from a light source such as a laser to propagate through an object such as a living body and detects a signal based on the propagation light to obtain information on the inside of the living body has been actively researched in a medical field. Photoacoustic imaging is known as one of such optical imaging techniques. Photoacoustic imaging is a technique of emitting a pulsed beam generated from a light source to an object, detecting acoustic waves generated from the tissues of a living body having absorbed the energy of light having propagated through and diffused into an object, and visualizing information on optical characteristic values inside the object. In this way, it is possible to obtain an optical characteristic value distribution inside the object (in particular, an optical energy absorption density distribution). For example, it is possible to image the image of blood vessels inside the living body in a non-invasive manner by using light having a wavelength that hemoglobin absorbs as the pulsed beam used for the photoacoustic imaging. Moreover, it is possible to image collagen and elastin under the skin by using a pulsed beam having another wavelength. Further, it is possible to emphasize a blood vessel image and image a lymphatic vessel by using a contrast agent.

A representative 3-dimensional visualization technique which uses photoacoustic imaging will be described. That is, the 3-dimensional visualization technique is a technique of detecting photoacoustic waves generated from a light absorber using an ultrasound transducer or the like disposed on a 2-dimensional surface and performing an image reconstruction operation to create 3-dimensional data related to optical characteristic values. This 3-dimensional visualization technique is referred to as photoacoustic tomography (PAT).

Further, in recent years, a photoacoustic microscope is gathering attention as an apparatus which enables visualization with high spatial resolution using the photoacoustic imaging technique. The photoacoustic microscope can acquire high-resolution images by focusing light or sound using an optical lens or an acoustic lens.

However, it is known that the visualization depth and the spatial resolution obtained by a photoacoustic apparatus such as a PAT apparatus or a photoacoustic microscope are in a trade-off relation. That is, PAT has such a property that the deeper the position of a tissue of a living body, of which the information is acquired, the lower the spatial resolution. Examples of the reasons therefor include the tendency of light to diffuse easily into a living body and the large attenuation of high-frequency photoacoustic waves generated inside the living body. Due to this property, the main use of a photoacoustic microscope having high spatial resolution is to visualize a light absorber inside the tissue present in a relatively shallow portion of a living body. For example, when hemoglobin in a blood is visualized using a photoacoustic microscope, a blood vessel present in the dermal layer of the tissue can be visualized.

Non-Patent Literature 1 discloses a photoacoustic microscope capable of imaging the image of blood vessels inside the tissue of a small animal with high resolution by using an acoustic lens.
Non-Patent Literature 1: Konstantin Maslov, Gheorghe Stoica, Lihong V. Wang, “In vivo dark-field reflection-mode photoacoustic microscopy”, OPTICS LETTERS, Mar. 15, 2005, Vol. 30, NO. 6.

### SUMMARY OF THE INVENTION

When the image of blood vessels present inside the tissue is imaged using a photoacoustic microscope, in order to acquire high-resolution images in an entire measurement region, the focusing position of a pulsed beam emitted to an object is preferably aligned at a depth at which the blood vessels in the skin run.

However, since the skin surface is generally not flat but has unevenness such as wrinkles or depression, when measurement is performed by scanning a 2-dimensional surface, the depths from the skin surface, of the focal point of the pulsed beam at respective measurement positions are different depending on the unevenness of the skin surface. Due to this, when imaging is performed, a blood vessel that is to be drawn deviates from the focal point of a pulsed beam whereby the run of the blood vessel is drawn discontinuously.

Moreover, in a method of scanning a focusing position of a pulsed beam over the entire 3-dimensional region to perform imaging, the measurement period increases dramatically as compared to a case of scanning over a 2-dimensional surface. Thus, a distortion of an image resulting from a motion of an object during the measurement period is not negligible. This can cause degradation in accuracy when measurement is sequentially performed at respective wavelengths using a plurality of pulsed beams of the respective wavelengths as in the case of calculating the oxygen saturation in the blood of an object, for example.

In this regard, the photoacoustic microscope disclosed in Non-Patent Literature 1, for example, does not take the degradation in the image accuracy resulting from the unevenness of the skin surface into consideration.

In view of the above problems, it is an object of the present invention to provide an object information acquiring apparatus capable of acquiring an object image with higher accuracy by taking the unevenness of the object surface into consideration.

In order to achieve the object, the present invention provides an aspect of an object information acquiring apparatus comprising: a light source that generates a first light beam; a receiving unit that includes an irradiating unit emitting the first light beam so as to be directed to a predetermined optical focusing region and an acoustic wave detecting unit detecting a first acoustic wave generated when the first light beam is emitted to an object; a scanning unit that performs relative movement between the object and the receiving unit so that the receiving unit follows a concave or convex shape of a surface of the object while the first light beam is emitted and the first acoustic wave is detected; and an acquiring unit that acquires characteristics information on the optical focusing region of the object based on a detection result obtained by the acoustic wave detecting unit.

According to another aspect of the present invention, an object information acquiring apparatus comprising: a light source that generates a light beam; a receiving unit that includes an irradiating unit emitting the light beam and an acoustic wave detecting unit detecting an acoustic wave generated from a predetermined acoustic focusing region when the light beam is emitted to an object; a scanning unit that performs relative movement between the object and the receiving unit so that the receiving unit follows a concave or convex shape of a surface of the object while the light beam is emitted and the acoustic wave is detected; and an acquiring unit that acquires characteristics information on the acoustic focusing region of the object based on a detection result obtained by the acoustic wave detecting unit is provided.

According to the aspects of the present invention, it is possible to provide an object information acquiring apparatus capable of acquiring an object image with higher accuracy by taking the unevenness of the object surface into consideration.

Further features of the present invention will become apparent from the following description of exemplary embodiments with reference to the attached drawings.

### BRIEF DESCRIPTION OF THE DRAWINGS

FIG. 1 is a block diagram illustrating Example 1 of an object information acquiring apparatus according to an embodiment of the present invention;

FIG. 2 is a timing chart illustrating an operation of the apparatus of Example 1;

FIG. 3 is a flowchart illustrating a data acquisition process of Example 1;

FIG. 4 is a flowchart illustrating another example of the data acquisition process of Example 1;

FIG. 5 is a block diagram illustrating Example 2 of an object information acquiring apparatus according to an embodiment of the present invention;

FIG. 6 is a timing chart illustrating an operation of the apparatus of Example 2;

FIG. 7 is a flowchart illustrating a data acquisition process of Example 2;

FIG. 8 is a schematic diagram illustrating a portion of the data acquisition process of Example 2;

FIG. 9 is a schematic diagram illustrating another example of the object information acquiring apparatus of Example 2;

FIG. 10 is a diagram illustrating Example 3 of an object information acquiring apparatus according to an embodiment of the present invention;

FIG. 11 is a timing chart illustrating an operation of the apparatus of Example 3;

FIG. 12 is a flowchart illustrating a data acquisition process of Example 3;

FIG. 13 is a diagram illustrating Example 4 of an object information acquiring apparatus according to an embodiment of the present invention; and

FIG. 14 is a flowchart illustrating a data acquisition process of Example 4.

### DESCRIPTION OF THE EMBODIMENTS

Hereinafter, embodiments of the present invention will be described in detail with reference to the drawings. The same constituent components will basically be denoted by the same reference numerals, and the description thereof will not be provided. However, detail arithmetic expressions, operation procedures, and the like disclosed below are to be appropriately changed according to the configuration and various conditions of an apparatus to which the present invention is applied, and the scope of the present invention is not limited to those described below.

A photoacoustic microscope which is an object information acquiring apparatus of the present invention includes an apparatus which uses a photoacoustic effect to emit light (electromagnetic waves) such as near-infrared rays to an object to receive an acoustic wave generated inside the object to thereby acquire object information as image data.

The object information acquired in the apparatus which uses the photoacoustic effect may be a generation source distribution of the acoustic wave generated by light irradiation, an initial acoustic pressure distribution inside the object, an optical energy absorption density distribution and an absorption coefficient distribution derived from the initial acoustic pressure distribution, or a concentration distribution of a substance that constitutes a tissue. Examples of the substance concentration distribution include an oxygen saturation distribution, a total hemoglobin concentration distribution, and an oxygenated or reduced hemoglobin concentration distribution.

Moreover, the characteristics information which is the object information at a plurality of positions may be acquired as a 2-dimensional or 3-dimensional characteristics distribution. The characteristics distribution is generated as image data indicating the characteristics information inside an object.

The acoustic wave referred in the present invention is typically an ultrasound wave and includes an elastic wave called a sound wave and an acoustic wave. The acoustic wave generated by the photoacoustic effect is referred to as an acoustic wave or a light-induced ultrasound wave.

### Example 1

In this example, an ultrasound-focusing photoacoustic microscope will be described as an example of an object information acquiring apparatus. In this example, the ultrasound focusing photoacoustic microscope is a photoacoustic microscope having such a configuration that a focusing region (corresponding to an optical focusing region) of a pulsed beam is broadened in relation to an ultrasound focusing region. However, the embodiments are not limited to this, and the present invention can be applied to an optical focusing photoacoustic microscope having such a configuration that a focusing region of a pulsed beam becomes smaller than a focusing region of an ultrasound wave.

(Overall Configuration of Apparatus)

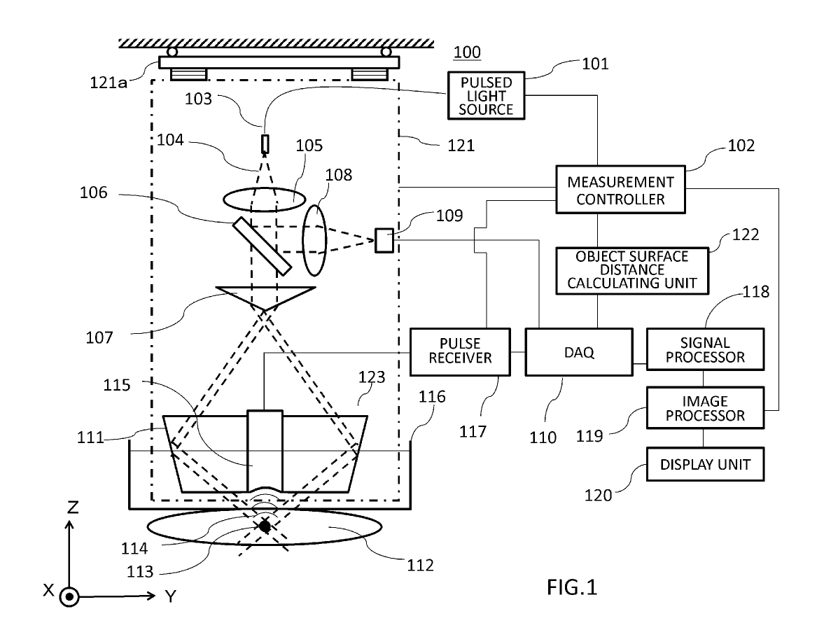

FIG. 1 is a block diagram illustrating Example 1 of an object information acquiring apparatus according to an embodiment of the present invention. An overall configuration of an object information acquiring apparatus 100 (hereinafter abbreviated to as an “apparatus 100”) of Example 1 will be described.

A pulsed light source 101 emits a pulsed beam under the control of a measurement controller 102 (corresponding to a scanning unit). The pulsed beam is guided to an optical system for emitting excitation light to a living body which is an object 112 through an optical fiber 103. In this example, this optical system includes a lens 105, a beam splitter 106, a conical lens 107, and a lens 108. A pulsed beam 104 output from the optical fiber 103 is collimated by the lens 105, and a portion of the collimated pulsed beam passes through the beam splitter 106 and another portion thereof is reflected by the beam splitter 106. The pulsed beam having passed through the beam splitter 106 is broadened in a ring form by the conical lens 107 and is incident to a mirror 111 (corresponding to an irradiating unit). On the other hand, the pulsed beam reflected by the beam splitter 106 is condensed by the lens 108 and is detected by a photodetector 109.

A data acquisition (DAQ) unit 110 performs A/D conversion on an electrical signal output by the photodetector 109 detecting the pulsed beam to generate a digital signal. The DAQ unit 110 stores the digital signal in an internal memory of the DAQ unit. The digital signal stored in this way can be used for correcting errors resulting from a variation in the light quantity of a photoacoustic signal which is a reception result of the photoacoustic wave generated when the pulsed beam is emitted to the object 112. Further, the digital signal can be used as a trigger signal for determining the measurement timing of the photoacoustic wave.

The conical lens 107 broadens the pulsed beam 104 in a ring form. The mirror 111 reflects the pulsed beam 104 broadened in the ring form to thereby condense the pulsed beam. The mirror 111 is formed using a transparent member such as glass as a base material, for example, and is configured to reflect the pulsed beam 104 at the boundary between the mirror 111 and the outside (air or water described later). Moreover, light reflectivity of the mirror 111 may be increased by depositing a metal film around the mirror 111. The position of the focal point of the condensed light is set so as to be inside the object 112 during measurement of the photoacoustic wave. In this case, since the pulsed beam 104 is condensed while maintaining the ring form, the pulsed beam 104 is not emitted directly to the surface of the object 112 immediately above the focal point. In this example, the lens 105, the conical lens 107, and the mirror 111 function as an optical unit that guides the pulsed beam 104 to the object 112.

The pulsed beam diffused into the object 112 is absorbed in a light absorber 113 such as the blood inside the object. The light absorber 113 has an optical absorption coefficient unique to a type thereof. The light absorber 113 generates a photoacoustic wave 114 by absorbing light. A transducer 115 (corresponding to an acoustic wave detecting unit) is provided near the center of the mirror 111 and is configured to detect the photoacoustic wave 114 to convert a change in acoustic pressure intensity thereof to an electrical signal (corresponding to a detection result). The transducer 115 is an ultrasound transducer that is sensitive to the frequency range of ultrasound waves, for example. The transducer 115 may include an acoustic lens. In the present embodiment, the transducer 115 includes an acoustic lens. By doing so, it is possible to condense an acoustic wave generated from the position of the focal point formed by the acoustic lens itself and detect the acoustic wave with high sensitivity. In the transducer 115, by setting the focal point (corresponding to an acoustic focusing region) of the acoustic lens to a predetermined position which is the focusing position of the pulsed beam condensed by the mirror 111, it is possible to detect the acoustic wave generated from the focusing position of the pulsed beam with high sensitivity. In this example, a receiving unit 123 is formed by integrating the mirror 111 and the transducer 115.

Water stored in a water tank 116 is present between the transducer 115 and the object 112, whereby acoustic impedance matching between the transducer 115 and the object 112 is realized. The acoustic matching impedance stored in the water tank 116 is not limited to water but another substance may be used. Moreover, a gel-state acoustic impedance material may be applied between the object 112 and the bottom of the water tank 116.

A pulse receiver 117 has a signal amplifier and is configured to receive an electrical signal obtained by the transducer 115 to amplify the intensity of the electrical signal with the aid of the signal amplifier. The DAQ unit 110 receives the electrical signal amplified by the signal amplifier and converts the electrical signal to a digital signal by A/D conversion. The DAQ unit has an internal memory and stores the converted digital signal in the internal memory as data. A signal processor 118 processes the data stored in the DAQ unit 110. An image processor 119 processes images based on the signal processing result obtained by the signal processor 118. A display unit 120 displays the image data based on the image processing result obtained by the image processor 119. The signal processor 118 and the image processor 119 may be configured as an integrated processor.

In this example, a member 121 surrounded by a one-dot chain line is mounted on a movable stage 121a that can scan in a 3-dimensional form. Moreover, the movable stage 121a is disposed above the member 121 surrounded by the one-dot chain line and the member 121 is hung from the movable stage 121a for convenience of explanation in FIG. 1. However, the embodiments are not limited to this. The movable stage 121a moves in a 3-dimensional form in relation to the object 112 to move to the positions of a focal point at which the pulsed beam 104 is condensed at the object 112 and the focal point of the transducer 115. By detecting the photoacoustic waves at the respective measurement positions scanned 3-dimensionally, it is possible to acquire photoacoustic signal data inside the object. However, the embodiments are not limited to this, and the object 112 may be moved in a 3-dimensional form in relation to the movable stage 121a so that the positions of the focal point of the pulsed beam 104 and the focal point of the transducer 115 are moved in the object 112.

In this example, the distance between the receiving-unit-side surface of the object 112 and the transducer 115 is measured. By doing so, the coordinate of the distance (corresponding to the measurement result) at the present point in time in an optical axis direction (in this example, the optical axis direction of the lens 105 and the same direction as the Z-axis direction) of an optical system that guides the pulsed beam on the surface of the object 112 to the object 112 is calculated. Moreover, a mechanism that determines the coordinate of the transducer in the optical axis direction at the measurement positions of the photoacoustic signals based on the coordinate is included.

The pulse receiver 117 outputs an instruction for transmitting an elastic wave to the object 112 to the transducer 115 based on the trigger signal generated by the measurement controller 102. The transducer 115 receives the instruction signal and outputs an elastic wave to the object 112. The transducer 115 receives an elastic wave reflected from the surface of the object to convert the elastic wave to an electrical signal, amplifies the signal intensity thereof, and transmits the amplified electrical signal to the DAQ unit. The DAQ unit converts the transmitted signal to a digital signal and transmits the digital signal to an object surface distance calculating unit 122.

The object surface distance calculating unit 122 calculates the distance between the surface of the object 112 and the transducer 115 using a delay period indicating how much the signal transmitted from the DAQ unit is delayed from the trigger signal generated by the measurement controller 102. The coordinate in the Z-axis direction, reflecting a concave or convex shape such as a wrinkle, a pimple, or a depression on an object surface, which is the coordinate in a coordinate system used for positioning the movable stage 121a is calculated from the distance. The measurement controller 102 determines the coordinate of the transducer 115 (or the movable stage 121a) in the Z-axis direction at the respective measurement positions of the photoacoustic signal based on the coordinate information in the Z-axis direction, reflecting an unevenness shape of the object surface. During photoacoustic measurement, the measurement controller 102 controls the movable stage 121a according to the determined coordinate information so that the movable stage is positioned at the determined coordinate. In this manner, the movable stage 121a of this example performs measurement while moving on a 2-dimensional curved surface, reflecting the unevenness shape of the object surface. That is, the movable stage of this example performs measurement while moving so as to follow the concave or convex shape of the surface of the object. A method of calculating the coordinate in the Z-axis direction of the object surface and a method of determining the coordinate in the Z-axis direction of the transducer 115 at the respective measurement positions based on the calculation value will be described later. Moreover, the measurement controller 102 controls the emission timing of the pulsed beam, controls the movable stage 121a, and controls data sampling of the DAQ unit 110.

In the above-described configuration, a configuration in which the focusing region (corresponding to one of focusing regions) of the pulsed beam 104 includes an ultrasound focal point (corresponding to the other focusing region) of the acoustic lens provided in the transducer 115 has been described. However, the embodiments are not limited to this, and this relation may be reverse as in an optical focusing photoacoustic microscope. That is, a focusing region in which the pulsed beam 104 is focused using an objective lens or the like may be included in an ultrasound focusing region of the acoustic lens of the transducer 115. That is, when the optical focusing photoacoustic microscope is used, since the size of the focal point of light determines the resolution of the photoacoustic microscope, it is possible to acquire a higher-resolution photoacoustic image.

(Operation Timing)

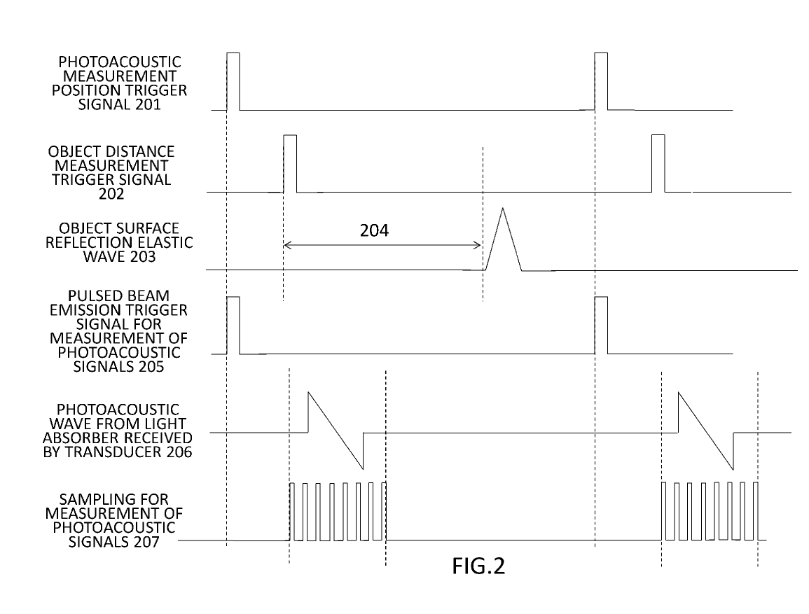

FIG. 2 is a timing chart illustrating the operation of the apparatus of Example 1. In this example, a trigger signal 201 generated by the measurement controller 102 is a pulsed signal. The measurement controller 102 generates a photoacoustic wave measurement position trigger signal 201. The rising timing of the trigger signal 201 is the time at which the focal point of the acoustic lens of the transducer 115 passes the photoacoustic measurement position set in advance by a user with scanning of the movable stage 121a. When the user sets the measurement position in advance, the user designates points at which the measurement positions on a 2-dimensional curved surface that is measured actually are projected on a 2-dimensional surface formed by two axes (X and Y-axes in FIG. 1) of the movable stage 121a orthogonal to the optical axis (Z-axis in FIG. 1) of the pulsed beam.

Specifically, a measurement pitch and a measurement range on the 2-dimensional surface are designated. An object distance measurement trigger signal 202 is generated based on the trigger signal 201. The trigger signal 202 determines a transmission timing of an elastic wave transmitted from the transducer 115 in order to measure the object surface distance. The trigger signal 202 may be generated in synchronism with the trigger signal 201 and a predetermined time shift may be present as in this example. That is, the object surface distance may not necessarily be measured at the same position as the photoacoustic measurement position. A reflection elastic wave 203 is an elastic wave reflected from the surface of the object 112 among the elastic waves transmitted from the transducer 115. The reflection elastic wave 203 is delayed by a delay period 204 in relation to the trigger signal 202 according to the distance between the surface of the object 112 and a sensor surface of the transducer 115. As will be described later, the object surface distance calculating unit 122 calculates the distance to the object surface based on the delay period 204. A pulsed beam emission trigger signal 205 is a signal that is synchronized with the start of measurement of the photoacoustic signal. The trigger signal 205 is synchronized with the photoacoustic measurement position trigger signal 201. A photoacoustic wave 206 is a photoacoustic wave which is excited inside the object by the pulsed beam emitted from the pulsed light source 101 at the timing indicated by the trigger signal 205 and reaches the transducer 115.

The photoacoustic wave 206 detected by the transducer 115 has an amount of delay in relation to the emission timing of the pulsed light source 101 corresponding to a period until the photoacoustic wave reaches the transducer 115 from a photoacoustic wave generation source. A sampling timing 207 defines the timing at which the photoacoustic wave having reached the transducer 115 is measured. Sampling starts with a delay from the signal 205 and sampling is performed over a width of time including at least the maximum and minimum peaks of the photoacoustic wave. However, if the DAQ unit 110 has a sufficient memory size, sampling may start in synchronism with emission of the pulsed light source 101 without delaying the sampling start time. Moreover, a measurement sampling frequency is preferably set to a sufficiently large frequency as much as possible that is at least twice the principal frequency of the generated photoacoustic wave.

It has been described that the trigger signal 202 for measuring the object distance may have a certain time shift in relation to the trigger signal 201. That is, this time shift is set so as not to overlap the time at which the reflection elastic wave 203 and the photoacoustic wave 206 reach the transducer 115. Moreover, a case in which the operation timing is set so that the scanning of the movable stage 121a is performed continuously without stopping has been described. However, the embodiments are not limited to this, and the respective measurements may be performed in a scanning method in which the movable stage 121a is stopped at positions at which photoacoustic measurement is executed or whenever the object distance is measured.

(Data Acquisition Process)

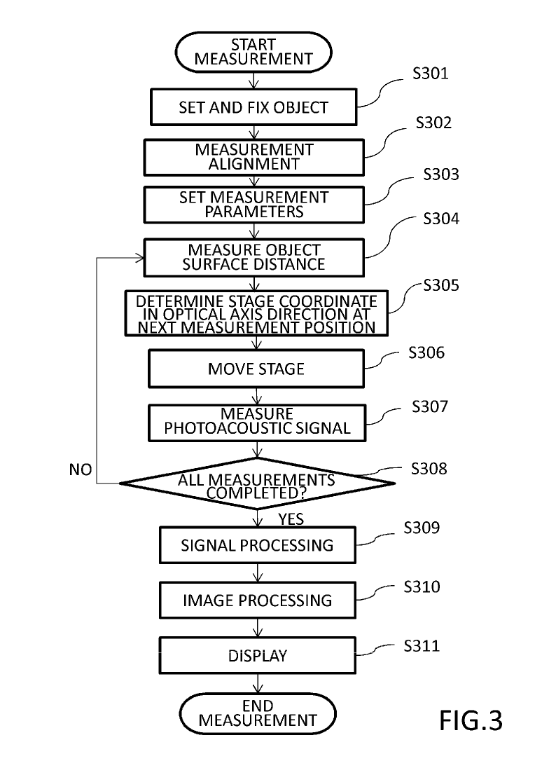

FIG. 3 is a flowchart illustrating a data acquisition process of Example 1. A method of acquiring a photoacoustic signal generated from the inside of the object 112 using the apparatus 100 described above and displaying images will be described in detail with reference to FIG. 3.

In step S301, the object 112 of which the photoacoustic image is measured by the apparatus 100 which is an ultrasound focusing photoacoustic microscope is set and fixed. In this case, a treatment of giving anesthetic, for example, may be performed appropriately so that the object 112 does not move during measurement. In step S302, the measurement controller 102 moves the ultrasound transducer 115 in the Z-axis direction to perform initial adjustment of the depth inside the object 112, of the acoustic focal point. As described above, the deeper the position inside the object, of the focal point of the pulsed beam, the more difficult to obtain a clear image. This is because the photoacoustic wave are scattered and attenuated by tissues inside the object or the pulsed beam 104 is diffused inside the object. Thus, the depth inside the object 112, of the acoustic focal point of the ultrasound transducer 115 is experimentally set according to the optical characteristics or the acoustic characteristics of the object 112 by taking this property into consideration.

In step S303, the measurement controller 102 sets measurement parameters for operating respective functional blocks. As for the measurement position at which a photoacoustic signal is acquired as one of the measurement parameters, as described above, an operator designates a measurement pitch and a measurement range projected on a 2-dimensional surface formed by the X and Y-axes in FIG. 1 by manual input or the like. The measurement range is a region in which the surface of an object is projected on an XY plane, for example, and may be individual regions obtained by dividing the region in a grid form. In this case, the measurement pitch may be the pitch between XY coordinates in each grid. Other examples of the measurement parameters include a storage sampling frequency and a storage period of photoacoustic signals per position, a scanning velocity and an acceleration of an automated stage, and an emission frequency, a light quantity, a wavelength, and the like of the pulsed light source 101.

In step S304, the distance between the surface of the object 112 and the transducer 115 is measured to calculate the coordinate of the object surface, in the Z-axis direction of an optical system that guides the pulsed beam to the object 112. The surface of the object 112 is a surface at a position facing the transducer 115. Specifically, this step is executed according to the following method.

First, the pulse receiver 117 transmits an instruction for transmitting an elastic wave to the object 112 to the transducer 115 based on the object distance measurement trigger signal 202 generated by the measurement controller 102. The transducer 115 receives an elastic wave reflected from the object surface and outputs an electrical signal and the pulse receiver 117 amplifies the intensity of the output signal. The DAQ unit converts the amplified signal to a digital signal and transmits the digital signal to the object surface distance calculating unit 122. The object surface distance calculating unit 122 calculates the distance between the surface of the object 112 and the transducer 115 based on the delay period 204 of the transmitted digital signal in relation to the object distance measurement trigger signal 202. The object surface distance calculating unit 122 calculates the distance between the object surface and the focusing position of the pulsed beam 104 by taking the known distance between the focal point of the pulsed beam and the sensor surface of the transducer 115 into consideration.

Here, the delay period 204 is defined as Δt [s] and the velocity of an elastic wave transmitted from the transducer 115 in the water in the water tank 116 is defined as ν [mm/s]. Moreover, the coordinate in the Z-axis direction of a movable stage 121a during measurement of the object surface distance, in a coordinate system for positioning the movable stage 121a is defined as Zₛₜ_{_}ₛ [mm]. In this case, the coordinate Zₛ [mm] in the Z-axis direction of the object surface in the positioning coordinate system is calculated using Equation (1) below.

Zₛ=Zₛₜ_{_}ₛ−((Δt)/2)×ν (1)

The elastic wave velocity ν in the water depends on the water temperature. Thus, the water temperature in the water tank 116 is measured using a thermometer and the velocity v is determined from the measured value. Moreover, in this example, it is assumed that the period in which an elastic wave propagates in the water occupies a large portion of the delay period 204 and the period in which the elastic wave propagates in the transducer 115 is negligibly short. When the period in which the elastic wave propagates in the transducer 115 is not negligible, an equation which takes the period and the velocity for the elastic wave to propagate through the transducer 115 into consideration may be used instead of Equation (1), and the subsequent process is the same as when Equation (1) is used.

Moreover, it is assumed that the central axis of the acoustic lens of the transducer 115 is parallel to the optical axis of the optical system that guides the pulsed beam 104 to the object 112. When the two axes are not parallel to each other, the subsequent process is similarly performed by correcting Equation (1) by taking an inclination between the two axes into consideration. The coordinate Zₛ [mm] is translated by a positive vector from the actual coordinate of the object surface in the coordinate system of the movable stage 121a, which does not affect the following discussion. Discussion will be made using this coordinate. That is, the actual coordinate of the object surface in the coordinate system of the movable stage 121a is a coordinate (Z_{const}+Zₛ) which is the addition of a coordinate Zₛ which is a calculation result and a constant reference value Z_{const}. However, since an optional position may be selected as the constant reference value Z_{const}, no particular problem occurs if the constant value Z_{const} is 0. That is, the coordinate Zₛ [mm] is translated by the positive vector Z_{const} from the actual coordinate (Z_{const}+Zₛ) of the object surface.

In step S305, the measurement controller 102 determines a coordinate Zₛₜ_{_}ₚₐ [mm] in the Z-axis direction of the movable stage 121a at the measurement position at which the next photoacoustic signal is acquired based on the calculated coordinate Zₛ [mm] calculated in step S304. This is determined so as to satisfy Equation (2) below.

L_{f1}−(Zₛₜ_{_}ₚₐ−Zₛ_{_}_{bar})=Δ (2)

Here, L_{f1} [mm], Δ [mm], and Zₛ_{_}_{bar} [mm] are known values. L_{f1} [mm] is the distance between the focal point of the acoustic lens of the transducer 115 and the sensor surface of the transducer 115. Moreover, Δ [mm] is the distance between the object surface and the focal point of the acoustic lens and a user can set the distance according to the position under the object surface at which a measurement target is present. The distance is set approximately between 0.05 and 0.2 [mm] when photoacoustic measurement is performed using the blood vessel under the epidermis of a person or the blood in a capillary blood as an imaging target. Moreover, Zₛ_{_}_{bar} [mm] is an approximate value of Zₛ [mm] at the measurement position of the next photoacoustic signal, and the following method can be used as a method of calculating the value. That is, a first method sets the coordinate Zₛ measured at the position closest to the measurement position at which the next photoacoustic measurement is performed as the value of Zₛ_{_}_{bar}. A second method sets a linear interpolation value of the coordinate Zₛ measured at a plurality of positions close to the measurement position at which the next photoacoustic measurement is performed as the value of Zₛ_{_}_{bar}. A third method sets a nonlinear interpolation value of the coordinate Zₛ measured at a plurality of positions close to the measurement position at which the next photoacoustic measurement is performed as the value of Zₛ_{_}_{bar}.

In step S306, the movable stage is moved to the measurement position of the next photoacoustic signal. First, the member 121 is moved in a direction (XY-axis direction) parallel to the object surface. By doing so, the transducer 115 is moved to a position in the XY-axis direction of the next measurement position. After that, the stage is moved in the Z-axis direction by referring to the coordinate of the stage in the Z-axis direction at the measurement position of the next photoacoustic signal determined in step S305 at the moved position on the XY plane. By doing so, the transducer 115 is moved to the next measurement position.

In step S307, the photoacoustic signal is measured. The photoacoustic signal is measured in the following order. The pulsed light source 101 emits a pulsed beam to the object 112 based on the pulsed beam emission trigger signal 205 generated by the measurement controller 102. The transducer 115 receives the photoacoustic wave 114 generated based on the light emission. The transducer 115 converts the acoustic pressure of the received photoacoustic wave to an electrical signal. The pulse receiver 117 amplifies the electrical signal. After that, the electrical signal amplified by the pulse receiver 117 is A/D-converted by the DAQ unit 110 and the A/D converted digital signal is stored in the internal memory of the DAQ unit 110. The data stored in the DAQ unit 110 is transmitted to the signal processor 118.

In step S308, it is determined whether measurement has been completed for all measurement ranges in the XY-axis direction set in step S303. If the measurement is not completed, the stage is moved in the XY-axis direction up to the next measurement range in which the object surface distance is measured. The object surface distance is measured again in step S304, and the stage is moved in the Z-axis direction and the photoacoustic measurement is performed again in the measurement range. The processes ranging from step S304 for measurement of the object distance to step S308 for determining whether the photoacoustic measurement has been completed are performed sequentially, and these processes are repeated until all measurements are completed.

When it is determined in step S308 that measurement in all measurement ranges has been completed, step S309 is executed. In this step, the signal processor 118 processes the electrical signal based on the photoacoustic signal acquired in the respective measurement ranges. Specific examples of the signal processing include deconvolution that takes the pulse width of the pulsed light source 101 into consideration and envelope detection. Moreover, when a specific frequency of noise added to signals is known in advance and this frequency can be separated from the principal frequency of a photoacoustic signal, a specific frequency component originating from noise may be removed. Moreover, such a process may be performed on waves transmitted directly from a photoacoustic wave source by being reflected from the surface of the object 112, the bottom of the water tank 116, and the like. That is, components and the like resulting from a photoacoustic wave arrived at the transducer 115 with such a delay are likely to become noise. Thus, noise components resulting from such a photoacoustic wave may be removed from a signal used for forming (reconstructing) an image by signal processing. Moreover, when the photoacoustic wave components generated from the surface of the object 112 are dominant, such noise components may be removed in step S308.

Step S310 is a step of performing image processing. In step S309, the image processor 119 creates voxel data based on the position on a scanning surface of the movable stage 121a and a signal intensity distribution in a depth direction of the object, of the processed photoacoustic signal obtained through signal processing. Image data for visualization is generated based on the voxel data. In this case, if a known artifact is present, the artifact may be removed from the voxel data. Moreover, when an oxygen saturation of a light absorber in an object is calculated, for example, voxel data in which an oxygen saturation value is stored may be created from voxel data of the photoacoustic signal intensity acquired at respective wavelengths of a plurality of pulsed beams. Besides this, when measurement is performed by setting the wavelength of a pulsed beam in order to use hemoglobin in the blood in an object as a main light absorber, the blood vessel image may be binarized and extracted from the acquired voxel data, for example.

In step S310, stage coordinates (stage coordinates in the Z-axis direction of respective measurement ranges) in the Z-axis direction of respective photoacoustic measurement positions, determined in step S305 may be reflected on the voxel data to image the stage coordinates. That is, when image data is created using only the photoacoustic signal measured in step S307, the unevenness shape of the object surface is not reflected on the image data. Thus, by reflecting the stage coordinate information in the Z-axis direction determined based on the measured the unevenness shape of the object surface on the voxel data as necessary, it is possible to express the actual shape of the object surface.

Step S311 is a step of displaying the voxel data created from the intensity distribution of the photoacoustic signal in step S310 according to a display method desired by the user. Examples of the display method include a method of displaying the cross-sections vertical to the X, Y, and Z-axes and a method of displaying the maximum, minimum, or mean value of the voxel data in the respective axis directions as a 2-dimensional distribution. Moreover, a user may set a region of interest (ROI) in the voxel data so that a user interface program displays statistical information on the shape of the light absorber in the region and oxygen saturation information.

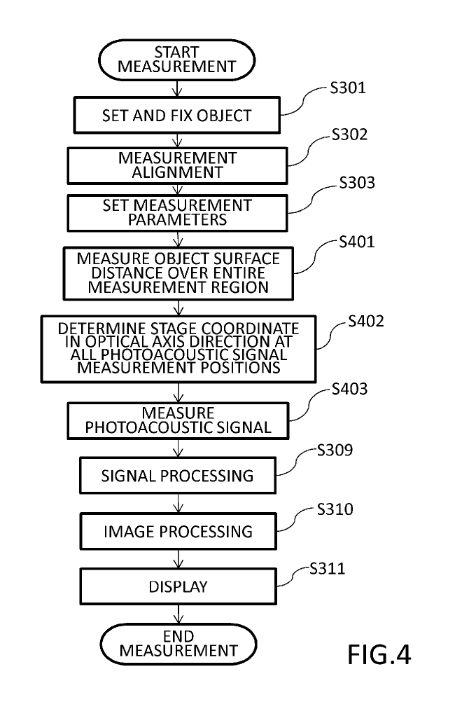

FIG. 4 is a flowchart illustrating another example of the data acquisition process of Example 1. In the above-described flow, the object distance is measured at the respective measurement positions, and the stage coordinate in the Z-axis direction at the next photoacoustic measurement position is determined. However, the coordinates Zₛ may be measured collectively in all measurement ranges which are all regions in which photoacoustic measurement is performed, the stage coordinates in the Z-axis direction at the respective position may be determined collectively, and then, photoacoustic measurement may be performed collectively.

Here, since the processes up to the measurement parameter setting step are the same as those of FIG. 3, the description thereof will not be provided. After that, in step S401, the coordinate Zₛ [mm] (the coordinate in a coordinate system for positioning the movable stage 121a) is measured in all measurement ranges of the region in which photoacoustic measurement is performed, set in step S303. Equation (1) is used in this calculation. In this case, photoacoustic measurement is not executed. Thus, a pulsed beam is not emitted to the object 112, or optical energy density per unit period is set to be smaller than that during actual photoacoustic measurement.

In step S402, a stage coordinate Zₛₜ_{_}ₚₐ [mm] in the Z-axis direction in respective measurement ranges in which photoacoustic measurement is performed is determined using Equation (2) based on coordinate distribution information in the Z-axis direction of the object surface obtained in step S401. In step S403, photoacoustic measurement is executed in all measurement ranges which are all measurement regions based on the value determined in step S402. The flow of the subsequent signal processing, image processing, and displaying processes is the same as that of FIG. 3, and the description thereof will not be provided.

<Others>

The configuration and the operation of the embodiment described above are examples only and may be changed. For example, light having a specific wavelength absorbed in a specific component among the components that constitute the object 112 may be used as the pulsed beam 104 emitted to the object 112. The pulse width of the pulsed beam 104 is several picoseconds to several hundreds of nanoseconds, and when the object 112 is a living body, it is preferable to use a pulsed beam having a width of several nanoseconds to several tens of nanoseconds. Although a laser is preferable as the pulsed light source 101 that generates the pulsed beam 104, a light-emitting diode, a flash lamp, or the like may be used instead of a laser. Various lasers such as a solid laser, a gas laser, a dye laser, or a semiconductor laser can be used as the laser of the pulsed light source 101. When a dye laser or an optical parametric oscillators (OPO) laser capable of changing an oscillating wavelength is used, a wavelength-based difference in an optical characteristic value distribution can be measured. A wavelength region between 400 nm and 1600 nm can be used for the wavelength of the pulsed light source 101, and light in the terahertz, microwave, and radio wave regions can be also used. When light of a plurality of wavelengths is used as the pulsed beam 104, the optical characteristics coefficients in a living body are calculated for the respective wavelengths, and the coefficients are compared with wavelength dependency unique to a substance (glucose, collagen, oxygenated and reduced hemoglobin, and the like) that constitutes a living body tissue. In this way, a concentration distribution of a substance that constitutes a living body may be imaged.

In this example, the movable stage 121a moves the transducer 115 that receives a photoacoustic signal and the focusing position of the pulsed beam 104 in the XY-axis direction and moves the same in the Z-axis direction for alignment. However, instead of mechanical movement in at least one direction, by changing the direction of light using a galvano mirror, it is possible to obtain the same advantage as the mechanical movement. Moreover, the same advantage may be obtained by partially moving the optical system that guides the pulsed beam to the object 112. Further, in this example, the distance to the object surface may be measured to control the coordinate in the Z-axis direction of the movable stage 121a by referring to the elastic wave reflected from the surface of the object 112. However, when layers having different acoustic impedances are present in an object, the movable stage 121a may be controlled by referring to a reflection elastic wave reflected from a boundary surface of these layers. For example, when the skin of an animal is the object, the skin is made up of layers of different acoustic impedances such as the epidermis, the dermis, and the subcutaneous fat. Thus, in this case, instead of referring to the shape of the skin surface, the coordinate in the Z-axis direction of the stage may be controlled by referring to the shape of the boundary surface between the epidermis and the dermis inside the skin or the boundary surface between the dermis and the subcutaneous fat.

By using the object information acquiring apparatus, it is possible to perform measurement by taking the unevenness of the object surface into consideration and to acquire high-resolution images in an entire measurement region.

### Example 2

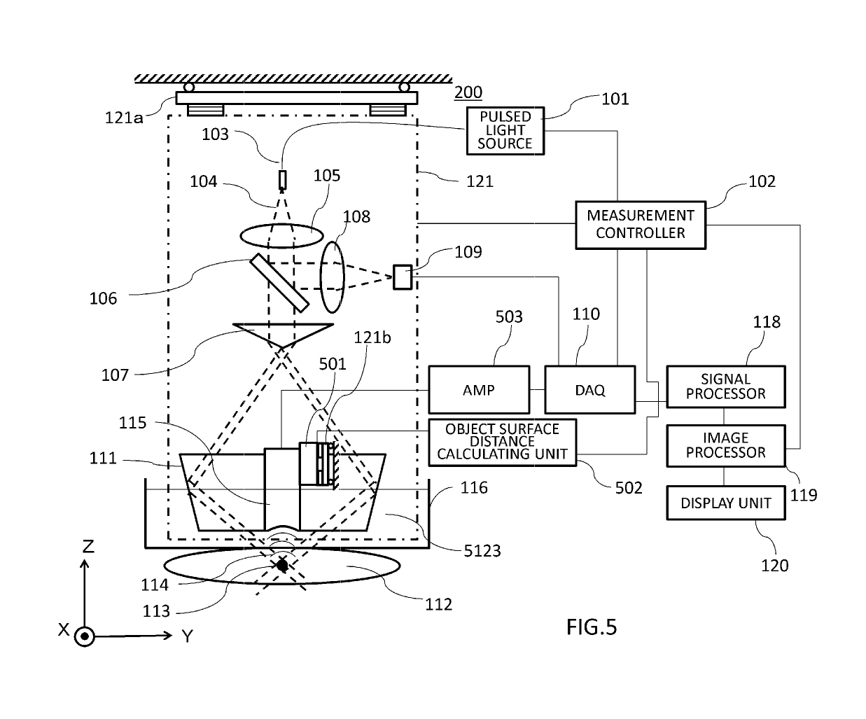

FIG. 5 is a block diagram illustrating Example 2 of an object information acquiring apparatus according to an embodiment of the present invention. The same constituent elements as those of Example 1 will be denoted by the same reference numerals, and the description thereof will not be provided. Hereinafter, an overall configuration of an object information acquiring apparatus 200 (hereinafter abbreviated to as an “apparatus 200”) of Example 2 will be described. In this example, a method different from that used in Example 1 is used as a method of measuring the unevenness shape of the object surface.

(Overall Configuration)

In this example, a length measuring method which uses an optical unit rather than an elastic wave transmitted from the transducer 115 is used as a method of measuring the unevenness shape of the object surface. An optical length measuring unit 501 has an optical unit and is provided adjacent to the transducer 115. The optical length measuring unit 501 is mounted on a movable stage 121a capable of scanning in a 3-dimensional form similarly to Example 1 together with the transducer 115 and an optical system for guiding the pulsed beam 104 to the object 112, surrounded by the frame of the member 121. An object surface distance calculating unit 502 receives distance information between the object 112 and the sensor of the optical length measuring unit 501, acquired by the optical length measuring unit 501 and calculates the coordinate in the Z-axis direction, reflecting the unevenness shape of the surface of the object 112 similarly to Example 1. The measurement controller 102 determines the coordinate in the optical axis direction of the movable stage 121a at respective positions at which photoacoustic measurement is performed based on the calculated distribution information on the unevenness shape of the object surface and controls the stage according to the determined value. In this example, a signal amplifier 503 amplifies the intensity of an electrical signal output when the transducer 115 receives the photoacoustic wave 114. The other constituent elements are the same as those of Example 1, and description thereof will not be provided.

The optical length measuring unit 501 is provided adjacent to the transducer 115, and the transducer 115, the optical length measuring unit 501, and the mirror 111 are integrated to form a receiving unit 5123. However, the embodiments are not limited to this, but only the optical length measuring unit 501 may be mounted to the movable stage 121b capable of scanning in a 2-dimensional or 3-dimensional form different from the above. However, in this case, the positions of a movable stage 121a on which the transducer 115 is mounted and a movable stage 121b on which an optical length measuring unit is mounted are preferably controlled based on the same coordinate system. In this way, the position at which signals are acquired by the transducer 115 can correspond to the position at which measurement is performed by the optical length measuring unit 501. Moreover, the movable stage 121b on which the optical length measuring unit 501 is provided is located next to the optical length measuring unit 501 for convenience of explanation in FIG. 5. However, the embodiments are not limited to this. In FIG. 5, it is shown that the movable stage 121b overlaps the pulsed beam 104 for convenience of explanation. However, the movable stage 121b should be located at the position that does not shade the pulsed beam 104. Moreover, a displacement meter or a shape measuring machine which uses a laser, a length measuring unit which uses an autofocus function of a camera or the like may be used as the optical length measuring unit 501. Further, as illustrated in FIG. 5, the body of the optical length measuring unit 501 is not necessarily mounted adjacent to the transducer 115, and a portion of an optical system for measuring the distance to the object 112 may be mounted. For example, only a mirror for reflecting a laser beam of a displacement meter which uses a laser may be mounted.

(Operation Timing)

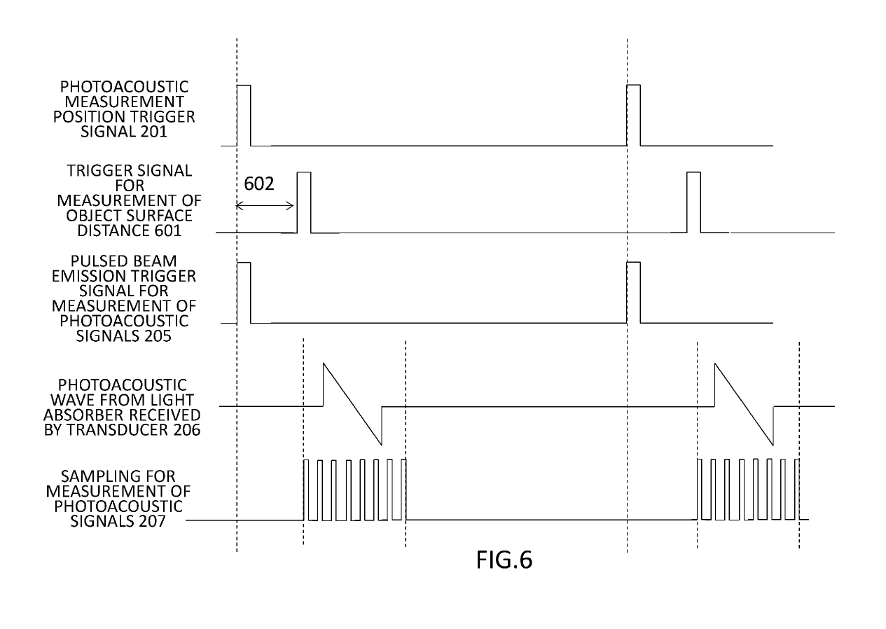

FIG. 6 is a timing chart illustrating the operation of the apparatus of Example 2. Since the measurement of photoacoustic signals is the same as that of Example 1, the description thereof will not be provided and the timing at which the distance to the object surface is measured by the optical length measuring unit 501 will be described. First, a case in which a wavelength region of light that the optical length measuring unit 501 uses for length measurement at least partially overlaps a wavelength region of light used for photoacoustic measurement, emitted by the pulsed light source 101 will be described. In this case, an object distance is measured at the rising timing of a object surface distance measurement trigger signal 601 delayed by a period 602 in relation to the photoacoustic wave measurement position trigger signal 201 and the pulsed beam emission trigger signal 205. The period 602 is longer than a pulse width of a pulsed beam generated at the rising timing of the trigger signal 205. In this way, it is possible to prevent the optical measurement by the optical length measuring unit 501 from affecting the photoacoustic measurement. However, the period 602 may not be provided for the timing at which the trigger signal 205 is not generated (that is, the timing at which the photoacoustic measurement is not executed) and the trigger signal 601 may be synchronized with the photoacoustic measurement position trigger signal 201. When the wavelength region of the light that the optical length measuring unit 501 uses for length measurement does not overlap the wavelength region of the light used by the pulsed light source 101, the optical measurement by the optical length measuring unit 501 may not affect the photoacoustic measurement even when both measurements are executed simultaneously. Thus, it is not necessary to provide the period 602 and the trigger signal 601 may be synchronized with the photoacoustic measurement position trigger signal 201 and the pulsed beam emission trigger signal 205.

(Data Acquisition Process)

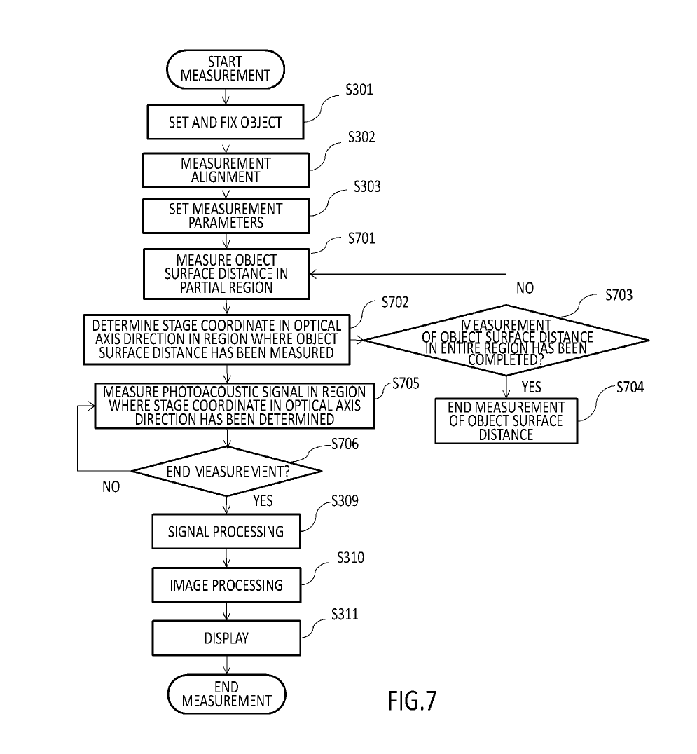

FIG. 7 is a flowchart illustrating a data acquisition process of Example 2. In this example, unlike Example 1, the optical length measuring unit 501 for measuring the unevenness shape of the surface of the object 112 and the transducer 115 that receives photoacoustic waves are provided at spatially different positions. Thus, when the measurement pitch at which photoacoustic measurement is performed is smaller than the distance between the optical length measuring unit 501 and the transducer 115, it is not possible to perform a measurement process in such an order that the object surface distance is measured and then the photoacoustic measurement is performed according to the measurement result. Thus, data acquisition is performed in the following process. In this example, the processes of steps S301 to S303 are the same as those of Example 1, and the description thereof will not be provided.

In step S701, the optical length measuring unit 501 measures the distance to the object surface in a measurement range in which photoacoustic measurement is performed, set in step S303. In this example, since an optical unit is used, when the coordinate Zₛ [mm] indicating the unevenness shape of the object surface is calculated, Equation (3) below is used rather than Equation (1) described in Example 1.

Zₛ=Zₛₜ_{_}ₛ−Lₛ (3)

However, similarly to Equation (2), the coordinate in the Z-axis direction of the movable stage 121a, 121b during measurement of the object surface is defined as Zₛₜ_{_}ₛ [mm]. Here, Lₛ [mm] is the distance from the measurement origin point of the optical length measuring unit 501 and the surface of the object 112, and the value measured by the optical length measuring unit 501 is substituted into Lₛ [mm]. In this example, it is assumed that the optical axis of the optical system that guides the pulsed beam 104 to the object is parallel to the optical axis of the optical length measuring unit 501 (the two axes are parallel to the Z-axis). When the two axes are not parallel to each other, the subsequent process is similarly performed by correcting Equation (3) by taking an inclination between the two axes into consideration.

In step S702, the measurement controller 102 determines the coordinate Zₛₜ_{_}ₚₐ [mm] in the Z-axis direction of the movable stage 121a, 121b at a position at which photoacoustic measurement is performed based on Zₛ calculated in step S701. In this example, Zₛₜ_{_}ₚₐ is calculated using Equation (4) below.

L_{f2}−Zₛₜ_{_}ₚₐ−Zₛ_{_}_{bar})=Δ (4)

Here, Δ [mm] and Zₛ_{_}_{bar} [mm] are the same values as described in Example 1. L_{f2} [mm] is the distance between the measurement origin point of the optical length measuring unit 501 and a plane including the coordinate in the Z-axis direction of the focal point of the acoustic lens of the transducer 115 and is a known value.

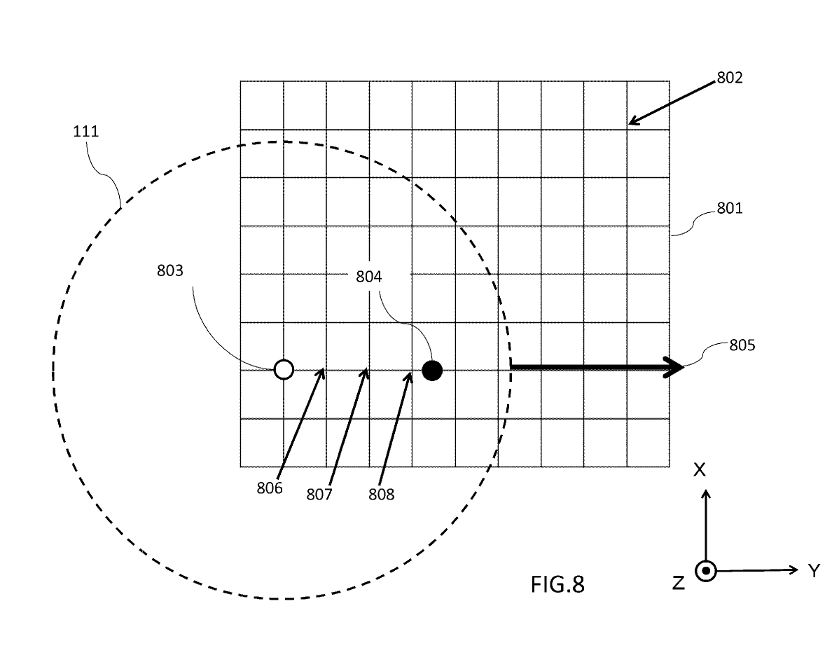

FIG. 8 is a schematic diagram illustrating a portion of the data acquisition process of Example 2. Steps S701 and S702 will be described in detail with reference to FIG. 8. FIG. 8 illustrates the measurement region of the object surface, the measurement position of the optical length measuring unit 501, and the position at which the photoacoustic measurement is performed by the transducer 115 when seen from a position facing the measurement region of the object surface. Grid points 802 included in an entire region 801 indicate the positions at which photoacoustic measurement is performed. The measurement region and the positions at which photoacoustic measurement is performed are set by the user in step S303. The mirror 111 reflects the pulsed beam 104 to condense the pulsed beam into the object, and the transducer 115 receives a photoacoustic wave generated from a white circle which is the focusing position 803 of the acoustic lens. A length measurement position 804 on the object surface, of the optical length measuring unit 501 is indicated by a black circle. The optical length measuring unit 501 measures the distance in a direction vertical to the drawing surface of FIG. 8 at the length measurement position 804 of the object surface. A scanning direction 805 indicates a scanning direction of the movable stage 121a, 121b. That is, the transducer 115 and the optical length measuring unit 501 move (corresponding to relative movement) in relation to each other in the direction 805 indicated by an arrow in the region 801. At the point in time illustrated in FIG. 8, photoacoustic measurement is not performed at positions 806, 807, and 808. However, the length measurement position 804 of the optical length measuring unit 501 has passed through the positions 806, 807, and 808, and the object surface distance has already been measured at the same positions as these three positions or a plurality of points near the three positions. A partial region in step S701 means a region in which the object surface is measured by the optical length measuring unit 501 before the photoacoustic measurement is performed. The partial region occurs when the distance between the focusing position 803 of the acoustic lens and the length measurement position 804 is larger than the measurement pitch at which photoacoustic measurement is performed. Moreover, the partial region occurs when the optical length measuring unit 501 is not a point sensor but a length measuring sensor capable of collectively acquiring the heights at a plurality of positions in 1-dimensional or 2-dimensional surface as a height distribution. When the measurement pitch of the photoacoustic measurement is larger than the distance between the positions 803 and 804, the measurement process described in FIG. 3 of Example 1 may be used.

In step S702, Zₛ_{_}_{bar} is calculated by the same method as used in Example 1 at the measurement positions 806, 807, and 808 of the photoacoustic signal and the coordinate Zₛₜ_{_}ₚₐ [mm] in the Z-axis direction of FIG. 5 is determined using Equation (4).

In step S703, it is determined whether the measurement of the surface distance of the object 112 has been completed in the entire region set in step S303. When the distance measurement is not completed, the flow proceeds to step S701 according to scanning of the movable stage 121a, 121b again and the distance measurement is performed similarly. When the distance measurement is completed, the flow proceeds to step S704 and the object surface distance measurement ends.

In step S705, photoacoustic measurement is executed in a region in which the coordinate Zₛₜ_{_}ₚₐ [mm] is determined in step S702. This method is the same as that of Example 1, and the description thereof will not be provided.

In step S706, it is determined whether photoacoustic measurement has been completed for the entire region set in step S303. When the measurement is not completed, the flow proceeds to step S705 again and the process is executed until the remaining measurement is completed.

The other data acquisition process is the same as that of Example 1, and the description thereof will not be provided. The process described above is executed according to a series of scanning operations of the movable stage 121a, 121b and the object surface distance measurement and the photoacoustic measurement are executed simultaneously in parallel. However, in this example, as described with reference to FIG. 4 in Example 1, the object surface distance may be measured over the entire measurement region. After that, the actual measurement may be performed after the coordinate in the Z-axis direction of the movable stage 121a, 121b at the photoacoustic measurement position is determined. This can be performed similarly to Example 1, and the description thereof will not be provided.

Various embodiments described in Section <Others> in Example 1 can be applied to this example. Moreover, in this example, although an example in which only one optical length measuring unit 501 is provided has been illustrated, a plurality of optical length measuring units may be provided. Further, an example in which the length can be measured at one point on the surface of the object 112 has been illustrated as the length measurement method of the optical length measuring unit 501. However, the embodiments are not limited to this, but a line sensor or a sensor capable of acquiring a height distribution of a 2-dimensional surface may be used.

<Others>

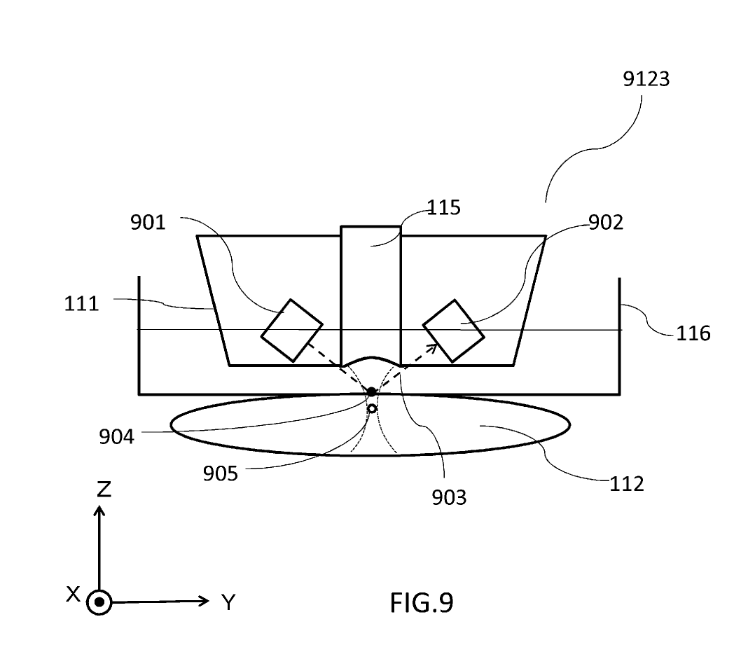

FIG. 9 is a schematic diagram illustrating another example of the object information acquiring apparatus of Example 2. A case in which a distance measurement position of the optical length measuring unit 501 and the focal point of the acoustic lens of the transducer 115 that receives photoacoustic waves are at spatially separated positions has been described. However, the embodiments are not limited to this, but can be applied to a case in which the two positions are at the same spatial position or neighbor each other. That is, FIG. 9 illustrates a portion in which the mirror 111, the transducer 115, and the water tank 116 of another example of the object information acquiring apparatus of Example 2 are provided at an enlarged scale. In this example, optical length measuring units 901 and 902 are used instead of the optical length measuring unit 501 as means for measuring a surface profile of the object 112. A receiving unit 9123 is formed by integrating the mirror 111, the optical length measuring units 901 and 902, and the transducer 115. These elements are based on the measurement principle which uses triangulation and are capable of measuring a displacement amount in the Z-axis direction of the surface of the object 112. The optical length measuring unit 901 emits a measurement laser beam 903 and the optical length measuring unit 902 receives a laser beam reflected and scattered from the surface of the object 112. In this case, a region 904 of the object surface to which the laser beam 903 is emitted is the position at which the displacement in the Z-axis direction is measured. An acoustic focusing region 905 in FIG. 9 is the focal point of the acoustic lens provided in the transducer 115. With such a configuration as illustrated in FIG. 9, the position 904 at which the unevenness shape in the Z-axis direction of the object surface is measured can be made identical to a position at which the acoustic focusing region 905 of the transducer 115 is projected on a plane vertical to the Z-axis. In such a case, measurement can be performed according to the process described in FIGS. 3 and 4 of Example 1 rather than the measurement process illustrated in FIG. 7. In FIG. 9, the optical length measuring units 901 and 902 are disposed inside the mirror 111. However, the embodiments are not limited to this, but the optical length measuring unit 901 and 902 may be disposed outside the mirror 111.

By using the object information acquiring apparatus, it is possible to perform measurement by taking the unevenness of the object surface into consideration and to acquire high-resolution images in an entire measurement region.

### Example 3

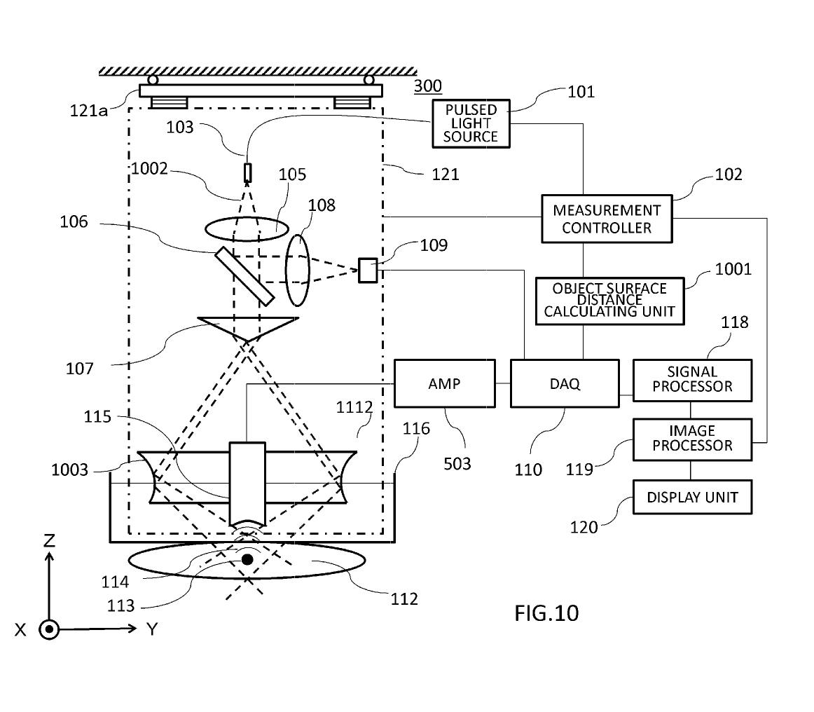

FIG. 10 is a block diagram illustrating Example 3 of an object information acquiring apparatus according to an embodiment of the present invention. The same constituent elements as those of Example 1 or 2 will be denoted by the same reference numerals, and the description thereof will not be provided. Hereinafter, an overall configuration of an object information acquiring apparatus 300 (hereinafter abbreviated to as an “apparatus 300”) of Example 3 will be described. In this example, the photoacoustic signal generated from the surface of the object 112 is used as a method of measuring the unevenness shape of the object surface unlike the methods used in Examples 1 and 2.

(Overall Configuration)

In this example, when photoacoustic measurement is performed, a pulsed beam 1002 for exciting a photoacoustic wave, emitted from a pulsed light source 1001 is guided so as to be emitted to an object surface immediately above the focal point of the acoustic lens of the transducer 115 positioned inside an object. Hereinafter, this illumination method will be referred to as bright visual-field illumination. In the apparatus 300, a convex mirror like a mirror 1003 is used to broaden the pulsed beam to realize bright visual-field illumination. However, the embodiments are not limited to this, but another illumination method may be used as long as bright visual-field illumination is realized. In this example, a receiving unit 1112 includes the mirror 1003 and the transducer 115.

The pulsed light source 1001 can emit light having such a wavelength in which the light is absorbed by the light absorber 113 as well as a surface segment of the object 112. When a region that overlaps a wavelength range in which light is absorbed by both the light absorber and the surface segment is present, the same wavelength in the wavelength range can be used. The constituent elements required for acquiring photoacoustic signals based on reception of photoacoustic waves and displaying images are the same as those of Example 2, and the description thereof will not be provided. As described above, in this example, when the surface profile of the object 112 is measured, photoacoustic waves generated from the object surface are used. In this case, the pulsed light source 1001 emits light having a wavelength in which the light is absorbed by a surface segment of the object 112, and the transducer 115 receives photoacoustic waves generated from the object surface by optical absorption. The signal amplifier 503 amplifies an electrical signal output from the transducer 115 as the result of reception of the photoacoustic waves.

The object surface distance calculating unit 1001 calculates the coordinate of the surface of the object 112 in the optical axis direction (the Z-axis direction) of the optical system that guides the pulsed beam to the object 112 based on the amplified signal. The measurement controller 102 inputs the calculated coordinate of the surface of the object 112 as data. The measurement controller 102 determines the coordinate in the Z-axis direction of the movable stage 121a to which the transducer 115 is fixed when the light absorber 113 inside the object is measured based on the data. The measurement controller 102 controls the movable stage 121a based on the determined coordinate.

In the above example, the single pulsed light source 1001 is configured to emit light of the same or different wavelengths in which the light is absorbed by the light absorber 113 and the surface segment of the object 112. However, the embodiments are not limited to this, but the pulsed light source may be a plurality of pulsed light sources capable of emitting light of different wavelengths.

(Operation Timing)

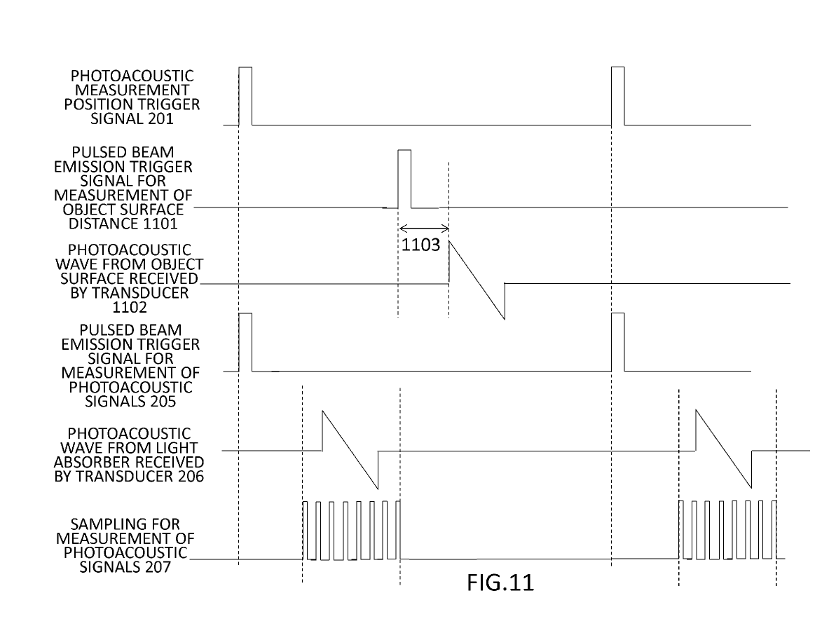

FIG. 11 is a timing chart illustrating the operation of the apparatus of Example 3. The trigger signal and sampling for photoacoustic measurement are the same as those described in FIG. 2, and the description thereof will not be provided. A trigger signal 1101 is a pulsed signal for determining an emission timing of a pulsed beam that generates a photoacoustic wave from the object surface in order to measure the distance to the surface of the object 112. A photoacoustic wave 1102 is generated from the object surface by the pulsed beam. The photoacoustic wave 1102 is received by the transducer 115 with a delay period 1103 from the rising timing of the trigger signal 1101. The period 1103 is a period corresponding to a propagation period of the photoacoustic wave propagating from the surface of the object 112 to the transducer 115. Here, the photoacoustic wave 1102 generated from the object surface and the photoacoustic wave 206 generated from the light absorber 113 are controlled so as not to overlap in time. In order to realize this timing relation, the rising timings of the pulsed beam emission trigger signal 1101 that generates a photoacoustic wave from the object surface and the pulsed beam emission trigger signal 205 that generates a photoacoustic wave from the light absorber 113 are shifted. However, when the wavelength of the pulsed beam for generation of photoacoustic waves from the surface of the object 112 is the same as the wavelength of the pulsed beam for generation of photoacoustic waves from the light absorber 113 and the respective photoacoustic waves do not overlap in time, the timings of the respective trigger signals may be synchronized with each other.

(Data Acquisition Process)

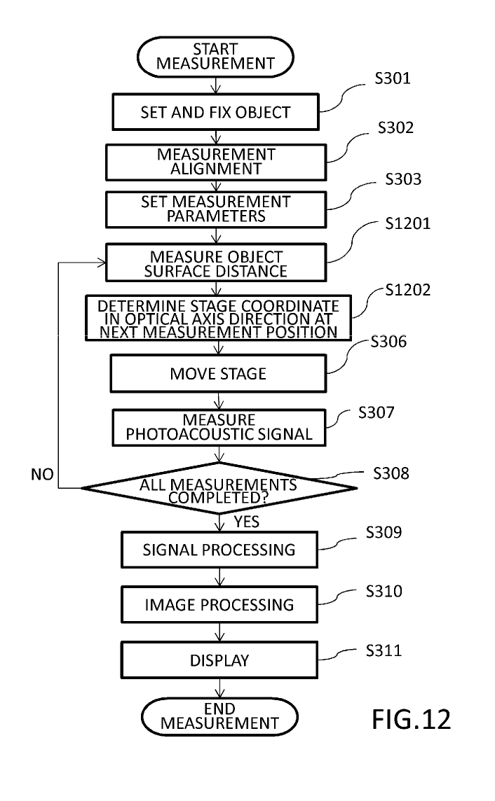

FIG. 12 is a flowchart illustrating a data acquisition process of Example 3. The processes of steps S301 to S303 are the same as those of Example 1, and the description thereof will not be provided. In step S1201, the transducer 115 receives a photoacoustic wave generated from the surface of the object 112. The object surface distance calculating unit 1001 measures the object surface distance based on the delay period 1103 (see FIG. 11). The object surface distance calculating unit 1001 calculates the coordinate Zₛ [mm] that reflects the unevenness shape of the object surface at a measurement position according to Equation (5) below.

Zₛ=Zₛₜ_{_}ₛ−ν×Δt (5)

Here, Zₛₜ_{_}ₛ [mm] is the coordinate in the Z-axis direction of the movable stage 121a when the object surface distance is measured. Δt [s] is the delay period 1103 which is a period elapsed until a photoacoustic wave generated from the object surface reaches the transducer 115 from the rising timing of the pulsed beam emission trigger signal 1101. ν [mm/s] is the velocity of a photoacoustic wave propagating in the water in the water tank 116. The velocity ν of the photoacoustic wave in the water depends on the water temperature as described in Example 1. Thus, the water temperature in the water tank 116 is measured using a thermometer and the velocity ν is determined from the measured value. Moreover, in this example, it is assumed that the period in which a photoacoustic wave propagates in the water occupies a large portion of the delay period 1103 and the period in which the photoacoustic wave propagates in the transducer 115 is negligibly short. When the period in which the photoacoustic wave propagates in the transducer 115 is not negligible, an equation which takes the period and the velocity for the photoacoustic wave to propagate through the transducer 115 into consideration may be used instead of Equation (5), and the subsequent process is the same as when Equation (5) is used. Moreover, in this example, it is assumed that the central axis of the acoustic lens of the transducer 115 is parallel to the optical axis of the optical system that guides the pulsed beam 104 to the object 112 (the two axes are parallel to the Z-axis). When the two axes are not parallel to each other, the subsequent process is similarly performed by correcting Equation (5) by taking an inclination between the two axes into consideration.

In step S1202, the measurement controller 102 determines the coordinate Zₛₜ_{_}ₚₐ [mm] in the Z-axis direction of the movable stage 121a at the measurement position at which the next photoacoustic signal is acquired based on the coordinate Zₛ [mm] calculated in step S1201. Since this coordinate can be determined so as to satisfy Equation (2) as described in Example 1, the detailed description thereof will not be provided. The other measurement processes can be executed similarly to Example 1, and the description thereof will not be provided. In the present embodiment, as described with reference to FIG. 4 in Example 1, the object surface distance may be measured over the entire measurement region. After that, the actual measurement may be performed after the coordinate in the Z-axis direction of the movable stage 121a at the photoacoustic measurement position is determined. This can be performed similarly to Example 1, and the description thereof will not be provided.

<Others>

Various embodiments described in Section <Others> in Example 1 can be applied to this example. By using the object information acquiring apparatus of Example 3, it is possible to perform measurement by taking the unevenness of the object surface into consideration. Thus, it is possible to acquire high-resolution images in an entire measurement region.

### Example 4

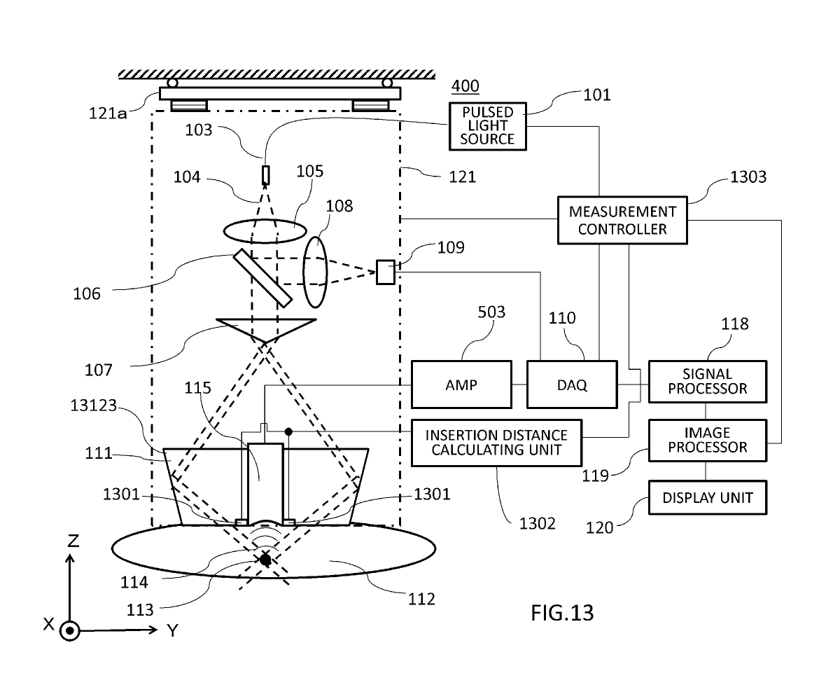

FIG. 13 is a block diagram illustrating Example 4 of an object information acquiring apparatus according to an embodiment of the present invention. Hereinafter, an overall configuration of an object information acquiring apparatus 400 (hereinafter abbreviated to as an “apparatus 400”) of Example 4 will be described. In Examples 1 and 3, a case in which the object 112 and the mirror 111 are not in contact with each other has been described mainly. In this example, a photoacoustic signal from a light absorber located at a deeper position inside an object is acquired.

In this example, the object 112 and the mirror 111 are in contact with each other. Although not illustrated in FIG. 13, liquid such as water, an acoustic impedance matching gel, or the like is applied to this contacting boundary surface in order to realize acoustic impedance matching. The position of the light absorber 113 inside the object changes depending on the pressure occurring in the contacting surface. Thus, a pressure sensor 1301 (corresponding to a pressure measuring unit) measures the pressure occurring in the contacting surface and a relative position of the transducer 115 and the mirror 111 in relation to the surface of the object 112 is controlled based on the pressure information. Specifically, an insertion distance of the mirror 111 in relation to the object surface is adjusted so as to be maintained constant. In this example, the insertion direction is an optical axis direction (the Z-axis direction) of the optical system that guides the pulsed beam 104 to the object 112. In this example, the pressure sensor 1301 is disposed at such a position that the optical path of the pulsed beam guided to the object 112 is not interrupted. Moreover, a strain gauge, a capacitive pressure sensor, a piezoelectric pressure sensor, and the like can be used as the pressure sensor. The pressure sensor 1301 transmits the measured pressure value to an insertion distance calculating unit 1302.

The insertion distance calculating unit 1302 calculates an insertion distance which is the distance of the mirror 111 inserted into the object surface in the Z-axis direction from the contact pressure value between the object surface and the mirror 111. In this example, a receiving unit 13123 is made up of the mirror 111, the pressure sensor 1301, and the transducer 115 and includes an electric wire for extracting an electrical signal from the pressure sensor 1301. The insertion distance calculating unit 1302 calculates the insertion distance by referring to a conversion table in which the pressure value of the contact surface is correlated with the insertion distance in the Z-axis direction of the object surface. The conversion table may be created in advance for the actual object 112 before photoacoustic measurement is performed and may be created in advance using a phantom that simulates the object 112. According to an exemplary method of creating the conversion table, the movable stage 121a is moved by a small amount in the Z-axis direction after the mirror comes into contact with the object surface, and the moved distance and the pressure value measured by the pressure sensor 1301 at that time are stored in advance in a memory in correlation. The memory may be provided in the insertion distance calculating unit 1302 and read appropriately during calculation and may be provided outside the insertion distance calculating unit 1302 and read appropriately. When the measured pressure value exceeds a certain pressure value designated by the user, the measurement controller 1303 may be controlled so as not to further insert into the object by taking the safety into consideration.

In this example, the data acquisition timings for photoacoustic measurement may be the same as those of the other examples. Moreover, the timings for measurement of the insertion distance from the object surface may be the same as the timings for measurement of the object distance of Example 2. Thus, the detailed description thereof will not be provided. In this example, since light is not emitted for measurement of the distance to the object unlike Example 2, it is not necessary to set the delay period 602.

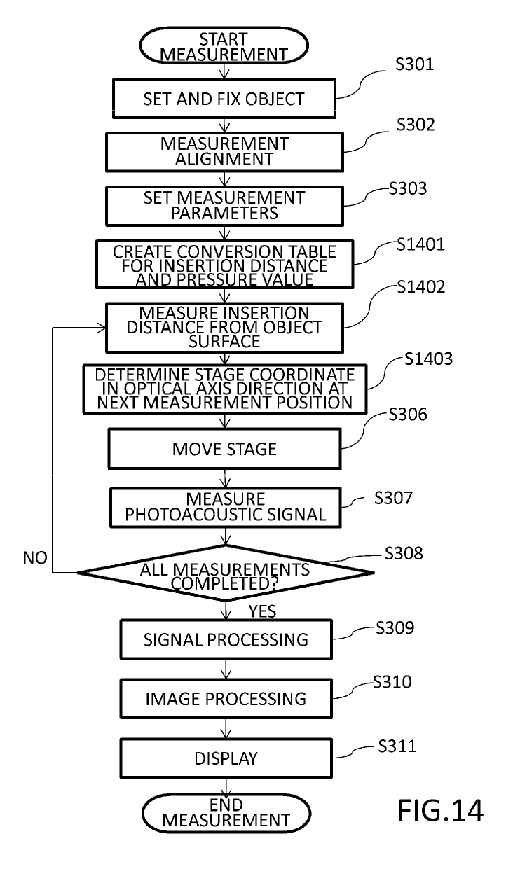

FIG. 14 is a flowchart illustrating a data acquisition process of Example 4. In this example, the processes of steps S301 to S303 are the same as those of Example 1. In step S1401, a conversion table for the insertion distance of the mirror 111 from the surface of the object 112 and the pressure value measured by the pressure sensor 1301 is created according to the above-described method. This conversion table may be created by a conversion table acquiring unit (not illustrated) and may be created by the insertion distance calculating unit 1302. The created conversion table is stored in a memory included in the insertion distance calculating unit 1302. In step S1402, the insertion distance calculating unit 1302 measures the insertion distance of the mirror 111 from the surface of the object 112. At the start of measurement of the insertion distance, the insertion distance is measured at the measurement position aligned in step S302 at the starting point of the measurement range set in step S303. Specifically, the insertion distance calculating unit 1302 calculates the insertion distance from the pressure measured by the pressure sensor 1301 by referring to the conversion table created in step S1401. Further, the coordinate Zₛ [mm] that reflects the unevenness shape of the object surface at that time is calculated based on Equation (6) below.

Zₛ=Zₛₜ_{_}ₛ+Lₚ (6)

Here, Zₛₜ_{_}ₛ [mm] is the coordinate in the Z-axis direction of the movable stage 121a when the object surface distance is measured. Moreover, Lₚ [mm] is an insertion distance of the object surface calculated from the contact pressure value calculated at that time.

In step S1403, the measurement controller 1303 determines the coordinate Zₛₜ_{_}ₚₐ [mm] based on the coordinate Zₛ that reflects the unevenness shape of the object surface, measured in step S1402. The Zₛₜ_{_}ₚₐ [mm] is the coordinate in the Z-axis direction of the movable stage 121a at the position at which the next photoacoustic measurement is performed. In this case, the coordinate Zₛₜ_{_}ₚₐ is calculated using Equation (7) below.

Zₛₜ_{_}ₚₐ=Zₛ_{_}_{bar}−Lₚᵢ (7)

Here, Zₛ_{_}_{bar} [mm] is the same as that described in Equation (2) of Example 1 and is an approximate value of Zₛ at the position at which the photoacoustic measurement is performed. Moreover, Lₚᵢ [mm] is an insertion distance of the object surface at the initial stage of measurement during measurement alignment in step S302. According to Equation (7), photoacoustic measurement can be performed while maintaining the insertion distance from the object surface to the initial value. The subsequent measurement processes are the same as those of Example 1, and the description thereof will not be provided. In this example, as described with reference to FIG. 4, the insertion distance from the object surface may be measured over the entire measurement region in advance. After that, the actual measurement may be performed after the coordinate in the Z-axis direction of the movable stage at the photoacoustic measurement position is determined. This can be performed similarly to Example 1, and the description thereof will not be provided.

<Others>

Various embodiments described in Section <Others> in Example 1 can be applied to this example. In the above-described configuration, the mirror 111 is in contact with the surface of the object 112. However, the embodiments are not limited to this, but in this example, the mirror may not be in contact with the object surface. That is, the pressure sensor 1301 may be separated from the mirror 111 and a positional relation thereof may be maintained constant. In this case, the pressure sensor is supported on a side closer to the object surface than the mirror 111 and makes contact with the object surface. The pressure sensor can measure the pressure on the contact surface so that the distance between the object surface and the mirror 111 or the transducer 115 is controlled so as to follow the unevenness of the object surface.

By using the object information acquiring apparatus, it is possible to perform measurement by taking the unevenness of the object surface into consideration and to acquire high-resolution images in an entire measurement region.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

A person having ordinary skill in the art could easily conceive a new system by appropriately combining various techniques of the respective examples. Thus, the system obtained by such combinations fall within the scope of the present invention.

The object information acquiring apparatus can be used as a medical image diagnostic device when the object is a living body substance. For example, in order to examine tumor or blood diseases and to observe the process of chemical treatments, it is possible to image an optical characteristic value distribution in the living body and a concentration distribution of substances that constitute a living body tissue obtained from the information. Moreover, the object information acquiring apparatus can be applied to non-destructive examination of non-living materials.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-240492, filed on Nov. 27, 2014, which is hereby incorporated by reference herein in its entirety.

What is claimed is:

1. An object information acquiring apparatus comprising:
a light source that generates a first light beam;
a receiving unit that includes an irradiating unit emitting the first light beam so as to be directed to a predetermined optical focusing region and an acoustic wave detecting unit detecting a first acoustic wave generated when the first light beam is emitted to an object;
a scanning unit that performs relative movement between the object and the receiving unit so that the receiving unit follows a concave or convex shape of a surface of the object while the first light beam is emitted and the first acoustic wave is detected; and
an acquiring unit that acquires characteristics information on the optical focusing region of the object based on a detection result obtained by the acoustic wave detecting unit.

2. The object information acquiring apparatus according to claim 1, wherein
the scanning unit measures a distance between the surface and the acoustic wave detecting unit and performs the relative movement based on a measurement result.

3. The object information acquiring apparatus according to claim 2, wherein
the acoustic wave detecting unit transmits an elastic wave to the surface when the scanning unit measures the distance and receives an elastic wave generated when the transmitted elastic wave is reflected from the surface, and
the scanning unit measures the distance based on a period elapsed until the elastic wave reflected from the surface is received after the elastic wave is transmitted to the surface.

4. The object information acquiring apparatus according to claim 2, further comprising:
a distance calculating unit that transmits an elastic wave to the surface when measuring the distance and receives an elastic wave generated when the transmitted elastic wave is reflected from the surface, wherein
the scanning unit measures the distance based on a period elapsed until the elastic wave reflected from the surface is received after the elastic wave is transmitted to the surface.

5. The object information acquiring apparatus according to claim 2, further comprising:
a distance calculating unit that emits a second light beam to the surface when measuring the distance and receives a second acoustic wave generated from the surface when the second light beam is emitted, wherein
the scanning unit measures the distance based on a period elapsed until the second acoustic wave is received after the second light beam is emitted.

6. The object information acquiring apparatus according to claim 2, further comprising:
a pressure measuring unit that measures pressure that the receiving unit applies to the object, wherein
the scanning unit measures the distance based on the measured pressure.

7. The object information acquiring apparatus according to claim 1, wherein
the acoustic wave detecting unit includes an acoustic lens, and
the acoustic lens that condenses an acoustic wave generated from an acoustic focusing region when the first light beam is emitted to the object.

8. The object information acquiring apparatus according to claim 7, wherein
one of the optical focusing region and the acoustic focusing region is included in the other one of the focusing regions.

9. An object information acquiring apparatus comprising:
a light source that generates a light beam;
a receiving unit that includes an irradiating unit emitting the light beam and an acoustic wave detecting unit detecting an acoustic wave generated from a predetermined acoustic focusing region when the light beam is emitted to an object;
a scanning unit that performs relative movement between the object and the receiving unit so that the receiving unit follows a concave or convex shape of a surface of the object while the light beam is emitted and the acoustic wave is detected; and
an acquiring unit that acquires characteristics information on the acoustic focusing region of the object based on a detection result obtained by the acoustic wave detecting unit.

10. The object information acquiring apparatus according to claim 1, wherein
the characteristics information is image data for forming an image.

11. The object information acquiring apparatus according to claim 10, further comprising:
a display unit that displays an image based on the image data.
